SEQUENCE LISTING (35 sequences)

SEQ ID NO: 1 - RNA, 11 nt, Artificial
  TLR13 activating minimal nucleid acid sequence
acggaannncc

SEQ ID NO: 2 - RNA, 11 nt, Artificial
  TLR13 activating minimal nucleid acid sequence
acggaaagacc

SEQ ID NO: 3 - RNA, 12 nt, Artificial
  TLR13 activating nucleid acid sequence
aacggaaagacc

SEQ ID NO: 4 - RNA, 32 nt, Artificial
  TLR13 activating sequence
gguuacccgcgacaggacggaaagaccccgug

SEQ ID NO: 5 - RNA, 23 nt, Artificial
  Nucleid acid sequence activating TLR13
caggacggaaagaccccguggag

SEQ ID NO: 6 - RNA, 19 nt, Artificial
  Nucleid acid activating TLR 13
ggacggaaagaccccgugg

SEQ ID NO: 7 - RNA, 17 nt, Artificial
  Nucleid acid sequence activating TLR13
gacggaaagaccccgug

SEQ ID NO: 8 - RNA, 12 nt, Artificial
  Nucleid acid sequence activating TLR13
gacggaaagacc

SEQ ID NO: 9 - RNA, 19 nt, Artificial
  Nucleid acid sequence activating TLR13
aaacggaaagaccaaaaaa

SEQ ID NO: 10 - RNA, 19 nt, Artificial
  Nucleid acid sequence activating TLR13
ggacggaaagaccccgugg

SEQ ID NO: 11 - RNA, 48 nt, Artificial
  Nucleid acid sequence activating both TLR13 and TLR 17
gguuacccgcgacaggacggaaagaccccguggagcuuuacuguagcc

SEQ ID NO: 12 - RNA, 33 nt, Artificial
  Nucleid acid sequence activating both TLR13 and TLR17
gacggaaagaccccguggagcuuuacuguagcc

SEQ ID NO: 13 - RNA, 11 nt, Artificial
  TLR13 inhibiting nucleid acid
ugccuunnngg

SEQ ID NO: 14 - RNA, 11 nt, Artificial
  TLR13 inhibiting nucleid acid
ugccuuucugg

SEQ ID NO: 15 - RNA, 48 nt, Artificial
  TLR13 inhibiting nucleid acid
ccaaugggcgcuguccugccuuucuggggcaccucgaaaugacaucgg

SEQ ID NO: 16 - RNA, 12 nt, Artificial
  TLR13 activating sequence
gccggaaagacc

SEQ ID NO: 17 - RNA, 12 nt, Artificial
  TLR13 activating sequence
gacggaacgacc

SEQ ID NO: 18 - RNA, 12 nt, Artificial
  TLR13 activating sequence
gacggaaaaacc

SEQ ID NO: 19 - RNA, 12 nt, Artificial
  TLR13 activating sequence
gacggaaagccc

SEQ ID NO: 20 - RNA, 12 nt, Artificial
  S. aureus 23S rRNA mimicking and derived oligoribonucleotides (ORNs)
gacggacagacc

SEQ ID NO: 21 - RNA, 12 nt, Artificial
  S. aureus 23S rRNA mimicking and derived oligoribonucleotides (ORNs)
gacgggaagacc

SEQ ID NO: 22 - RNA, 12 nt, Artificial
  S. aureus 23S rRNA mimicking and derived oligoribonucleotides (ORNs)
gacgaaaagacc

SEQ ID NO: 23 - RNA, 12 nt, Artificial
  S. aureus 23S rRNA mimicking and derived oligoribonucleotides (ORNs)
aaaagaaagaaa

SEQ ID NO: 24 - RNA, 12 nt, Artificial
  S. aureus 23S rRNA mimicking and derived oligoribonucleotides (ORNs)
gacggaaagaaa

SEQ ID NO: 25 - RNA, 12 nt, Artificial
  S. aureus 23S rRNA mimicking and derived oligoribonucleotides (ORNs)
aaaagaaagacc

SEQ ID NO: 26 - RNA, 12 nt, Artificial
  S. aureus 23S rRNA mimicking and derived oligoribonucleotides (ORNs)
gacggaaagaca

SEQ ID NO: 27 - RNA, 12 nt, Artificial
  S. aureus 23S rRNA mimicking and derived oligoribonucleotides (ORNs)
aaaggaaagacc

SEQ ID NO: 28 - RNA, 12 nt, Artificial
  S. aureus 23S rRNA mimicking and derived oligoribonucleotides (ORNs)
gacccaaagagg

SEQ ID NO: 29 - RNA, 12 nt, Artificial
  S. aureus 23S rRNA mimicking and derived oligoribonucleotides (ORNs)
gacggaaagacc

SEQ ID NO: 30 - RNA, 12 nt, Artificial
  S. aureus 23S rRNA mimicking and derived oligoribonucleotides (ORNs)
gacggaaagacc

SEQ ID NO: 31 - RNA, 20 nt, Artificial
  S. aureus 23S rRNA mimicking and derived oligoribonucleotides (ORNs)
ccgacacagguagucaagau

SEQ ID NO: 32 - RNA, 15 nt, Artificial
  S. aureus 23S rRNA mimicking and derived oligoribonucleotides (ORNs)
gcaccucgangucgc

SEQ ID NO: 33 - RNA, 12 nt, Artificial
  S. aureus 23S rRNA mimicking and derived oligoribonucleotides (ORNs)
gacggaaagacc

SEQ ID NO: 34 - RNA, 10 nt, Artificial
  S. aureus 23S rRNA mimicking and derived oligoribonucleotides (ORNs)
ggaaagaccn

SEQ ID NO: 35 - RNA, 11 nt, Artificial
  truncated version of SED ID NO 8
acggaaagacc

(19) United States
(12) Patent Application Publication
(10) Pub. No.: US 2017/0137820 A1
(43) Pub. Date: May 18, 2017

(54) AGONISTS AND ANTAGONISTS OF TOLL-LIKE RECEPTOR (TLR) 13

(71) Applicant: Bavarian Nordic A/S, Kvistgaard (DK)

(72) Inventors: Carsten Kirschning, Essen (DE); Stefan Bauer, Marbury-Michelbach (DE); Hubertus Hochrein, Munich (DE)

(73) Assignee: Bavarian Nordic A/S, Kvistgaard (DK)

(21) Appl. No.: 15/418,248

(22) Filed: Jan. 27, 2017

Related U.S. Application Data
(62) Division of application No. 14/377,035, filed on Aug. 6, 2014, now Pat. No. 9,556,439.

(51) Int. Cl. C12N 15/117; C12N 15/113; A61K 39/39; A61K 39/085; A61K 39/09

(52) U.S. Cl. CPC C12N 15/117; A61K 39/085; A61K 39/092; A61K 39/39; C12N 15/1138; C12N 2310/17; C12N 2320/31; C12N 2330/10; A61K 2039/55561

(57) ABSTRACT

The present invention relates to the field of immunology. The present invention provides agonists and antagonists of Toll-like receptor (TLR) 13. In particular, the present invention provides TLR13 activating and inhibiting nucleic acids, and provides such nucleic acids for use as pharmaceutical agents. The present invention further provides in vitro methods using such nucleic acids.

This application is a divisional application of U.S. Ser. No. 14/377,035, filed on Aug. 6, 2014 (now U.S. Pat. No. 9,556,439), which is a National Phase application under 35 U.S.C. §371 of International Application No. PCT/EP2013/00392, filed Feb. 8, 2013, and claims the benefit under 35 U.S.C. §119(e) of U.S. Provisional Patent Application 61/597,063 filed Feb. 9, 2012, the disclosures of which are incorporated by reference herein in their entirety.

### FIELD OF THE INVENTION

The present invention relates to the field of immunology. The present invention provides agonists and antagonists of Toll-like receptor (TLR) 13. In particular, the present invention provides TLR13 activating and inhibiting nucleic acids, and provides such nucleic acids for use as pharmaceutical agents. The present invention further provides in vitro methods using such nucleic acids.

### BACKGROUND OF THE INVENTION

The immune system recognises pathogens and potential danger with pattern recognition receptors (PRR). This sensing induces innate and adaptive immune responses and often is the prerequisite of a timely and effective immune defence against pathogens. However, an excessive immune response is dangerous and potential fatal as in the case of sepsis.

Among different families of PRR the Toll-like receptors (TLRs) have been recognized as very important for the activation of several innate and adaptive immune responses. Within the last years many different endogenous as well as artificial agonists and ligands for TLRs have been identified including agonists for TLR1, TLR2, TLR3, TLR4, TLRS, TLR6, TLR7, TLR8, TLR9, and TLR11. For example, certain viral RNA as the agonists for TLR7 and TLR8 have been identified¹⁷. This knowledge allowed the use of TLR-ligands as adjuvants for the induction of superior immune responses in vaccines or in different therapies against infectious diseases and cancer. On the other hand this knowledge made it possible to interfere with unwanted immune responses by blocking TLR recognition. Examples of unwanted or exaggerated immune responses are autoimmune diseases, infection and sepsis. The immune responses induced by different TLR-agonists differ greatly, thus the identification of new TLR-agonists allows a more fine tuned application, e.g. as adjuvants.

Whereas TLR2, TLR4 and TLR9 are major host sensors of Gram-negative bacteria, TLR2 and TLR7 are believed to be central detectors of Gram-positive bacteria.¹ Recognition of Gram-positive bacteria by TLR2 or TLR7 occurs via their lipoproteins, RNA and DNA, respectively²⁻⁵. However, the role of additional TLRs or other classes of PRRs such as C-type lectins, RIG-I-like helicases, or nucleotide binding domain—and leucine-rich repeat—containing proteins for detection of gram-positive bacteria is unclear. In particular, among the 13 different TLRs described in man and mouse the ligands for TLR10, TLR12 and TLR13 are unknown so far.

Thus, there is still a need for elucidating the role of TLRs in host protection from bacterial infection. In particular, it is an object to provide tools for targeting such TLRs, and methods and medical applications using them.

### SUMMARY OF THE INVENTION

The object of the present invention is solved by a nucleic acid comprising or consisting of a sequence of SEQ ID NO: 1 (ACGGAAXXXCC) or a variant thereof for use as a pharmaceutical agent, where ‘X’ signifies any nucleotide (e.g., A, C, G, T, or U). In the appended Sequence listing ‘X’ is ‘N’ because of the prescribed WIPO ST.25 standard.

In one embodiment the nucleic acid comprises or consists of a sequence of SEQ ID NO: 2 (ACGGAAAGACC) or a variant thereof.

In one embodiment, the nucleic acid comprises or consists of a sequence selected from the group consisting of SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO) or a variant thereof.

In a preferred embodiment, the nucleic acid comprises or consists of a sequence of SEQ ID NO: 10 (Sa19PSO) or a variant thereof.

In one embodiment, the nucleic acid comprises or consists of SEQ ID NO: 11 (SaIII) or SEQ ID NO: 12 (SaIIId5) or a variant thereof.

In one embodiment, the nucleic acid is for use as a pharmaceutical agent for activating Toll-like receptor (TLR) 13 expressing cells in a subject.

In one embodiment, the nucleic acid is for use as a pharmaceutical agent for stimulating an immune response in a TLR13 expressing subject.

In one embodiment, the nucleic acid is for use as a pharmaceutical agent for (use in a method of) stimulating an immune response in a non-primate subject, preferably in a non-human subject.

In one embodiment, the pharmaceutical agent is an immunostimulant.

In one embodiment, the pharmaceutical agent is an adjuvant.

In a preferred embodiment, the adjuvant is for vaccination against bacterial infection. Also, in another preferred embodiment, the adjuvans is for use in a method for vaccination against bacterial infection.

In one embodiment, the pharmaceutical agent is for treating an infection by a Gram-positive or Gram-negative bacterium resistant to one or more antibiotics. Also, in another embodiment the pharmaceutical agent is for use in a method of treating an infection by a Gram-positive or Gram-negative bacterium resistant to one or more antibiotics

In a preferred embodiment, the infection is a systemic or a local infection.

In another preferred embodiment, the Gram-positive or Gram-negative bacterium is resistant to one or more antibiotics of the macrolide, lincosamide, and streptogramin (MLS) group.

In a more preferred embodiment, the Gram-positive or Gram-negative bacterium is resistant to erythromycin.

In one embodiment, the Gram-positive bacterium is Staphylococcus aureus or Streptococcus pneumoniae.

The object of the present invention is further solved by a pharmaceutical composition comprising a nucleic acid comprising or consisting of a sequence of SEQ ID NO: 1 (ACGGAAXXXCC) or SEQ ID NO: 2 (ACGGAAAGACC) or a variant thereof.

In one embodiment, the pharmaceutical composition comprises a nucleic acid comprising or consisting of a sequence selected from the group consisting of SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), SEQ ID NO: 10 (Sa19PSO), SEQ ID NO: 11 (SaIII), and SEQ ID NO: 12 (SaIIId5) or a variant thereof, preferably comprising or consisting of a sequence of SEQ ID NO: 10 (Sa19PSO) or a variant thereof.

In one embodiment, the pharmaceutical composition is for systemic or local administration.

The object of the present invention is further solved by a nucleic acid comprising or consisting of a sequence of SEQ ID NO: 13 (UGCCUUXXXGG) or a variant thereof for use as a pharmaceutical agent.

In one embodiment, the nucleic acid comprises or consists of a sequence of SEQ ID NO: 14 (UGCCUUUCUGG) or a variant thereof.

In one embodiment, the nucleic acid comprises or consists of a sequence of SEQ ID NO: 15 (SaIIIas) or a variant thereof.

In one embodiment, the nucleic acid is for use as a pharmaceutical agent for inhibiting TLR13 expressing cells in a subject.

In one embodiment, the nucleic acid is for use as a pharmaceutical agent for inhibiting an immune response in a TLR13 expressing subject.

In one embodiment, the nucleic acid is for use as a pharmaceutical agent for inhibiting an immune response in a non-primate subject, preferably in a non-human subject.

In one embodiment, the pharmaceutical agent is an immunosuppressant.

In one embodiment, the pharmaceutical agent is for treating septic syndrome induced by bacteria.

In one embodiment, the pharmaceutical agent is for treating a local bacterial infection.

In a preferred embodiment, the pharmaceutical agent is a Gram-positive or Gram-negative bacterium.

In a preferred embodiment, the Gram-positive bacterium is Staphylococcus aureus or Streptococcus pneumoniae.

The object of the present invention is further solved by a pharmaceutical composition comprising a nucleic acid comprising or consisting of a sequence of SEQ ID NO: 13 (UGCCUUXXXGG) or SEQ ID NO: 14 (UGCCUUUCUGG) or a variant thereof.

In one embodiment, the pharmaceutical composition comprises a nucleic acid comprising or consisting of a sequence of SEQ ID NO: 15 (SaIIIas) or a variant thereof.

In one embodiment, the pharmaceutical composition is for systemic or local administration.

The object of the present invention is further solved by an in vitro method for activating TLR13 expressing cells, or for inducing cytokine and/or NO release from TLR13 expressing cells, comprising the step of contacting the cells with a nucleic acid comprising or consisting of a sequence of SEQ ID NO: 1 (ACGGAAXXXCC) or a variant thereof.

The object of the present invention is further solved by an in vitro method for studying TLR13 mediated cell activation, comprising the steps of:

(a) contacting TLR13 expressing cells, or cells to be examined for TLR13 expression, with a nucleic acid comprising or consisting of a sequence of SEQ ID NO: 1 (ACGGAAXXXCC) or a variant thereof;
(b) determining cytokine and/or NO release from the cells.

In one embodiment of the in vitro method for studying TLR13 medicated cell activation further comprises the step of contacting the cells with an inhibitor of TLR13 mediated cell activation prior to step (a).

In a preferred embodiment, the inhibitor is a nucleic acid comprising or consisting of an antisense sequence being complementary to the sequence of SEQ ID NO: 1 (ACGGAAXXXCC) or SEQ ID NO: 2 (ACGGAAAGACC) or a variant thereof.

In another preferred embodiment, the inhibitor is a nucleic acid comprising or consisting of an antisense sequence of SEQ ID NO: 13 (UGCCUUXXXGG) or SEQ ID NO: 14 (UGCCUUUCUGG) or a variant thereof.

In a more preferred embodiment, the inhibitor is a nucleic acid comprising or consisting of an antisense sequence of SEQ ID NO: 15 (SaIIIas) or a variant thereof.

In one embodiment of the above in vitro methods the nucleic acid comprises or consists of a sequence selected from the group consisting of SEQ ID NO: 2 (ACGGAAAGACC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO) or a variant thereof.

In one embodiment of the above in vitro methods the nucleic acid comprises or consists of SEQ ID NO: 11 (SaIII) or SEQ ID NO: 12 (SaIIId5) or a variant thereof.

In one embodiment of the above in vitro methods the cytokine is selected from the group consisting of IFN-λ, IL-6, IL-12p70, and TNFα.

In one embodiment of the above in vitro methods the TLR13 expressing cells are macrophages or conventional dendritic cells (cDCs).

In a preferred embodiment of the above in vitro methods the cytokine is IL-6 or TNFα, and the TLR13 expressing cells are macrophages.

In another preferred embodiment of the above in vitro methods the cytokine is IL-12p70, and the TLR13 expressing cells are cDCs.

In one embodiment of the above in vitro methods the cDCs are TLR23479^{-/-} eCD8^{high} cDCs or signal regulatory protein α (Sirp)^{high} cDCs.

The object of the present invention is further solved by a use of a nucleic acid comprising or consisting of a sequence selected from the group consisting of a sequence of SEQ ID NO: 1 (ACGGAAXXXCC), SEQ ID NO: 2 (ACGGAAAGACC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO), SEQ ID NO: 11 (SaIII), SEQ ID NO: 12 (SaIIId5), SEQ ID NO: 13 (UGCCUUXXXGG), SEQ ID NO: 14 (UGCCUUUCUGG), and SEQ ID NO: 15 (SaIIIas) or a variant thereof for studying TLR13 expressing cells or TLR13 mediated signal transduction.

The object of the present invention is further solved by a nucleic acid comprising or consisting of a sequence selected from the group consisting of SEQ ID NO: 1 (ACGGAAXXXCC), SEQ ID NO: 2 (ACGGAAAGACC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO), SEQ ID NO: 11 (SaIII), SEQ ID NO: 12 (SaIIId5), SEQ ID NO: 13 (UGCCUUXXXGG), SEQ ID NO: 14 (UGCCUUUCUGG), and SEQ ID NO: 15 (SaIIIas) or a variant thereof.

The term “variant” of a defined nucleic acid means a nucleic acid being modified compared to the defined nucleic acid, e.g. in nucleotide sequence (e.g., by deletions, additions or exchanges/mutations/substitutions of nucleotides) or in individual nucleotides (e.g., by methylation of a nucleotide). In particular, the term shall include modifications of a defined nucleic acid aiming at stabilisation, e.g. by thioate or phosphorothioate modification. The term “variant” encompasses nucleic acids comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or more additional nucleotides compared to a nucleic acid of defined sequence (e.g., SEQ ID NO:2), nucleic acids comprising 1, 2, 3, 4, 5, or more fewer nucleotides compared to a nucleic acid of defined sequence (e.g., SEQ ID NO:2), nucleic acids comprising 1, 2, or more modified nucleotides (e.g., methylated nucleotides) compared to a nucleic acid of defined sequence (e.g., SEQ ID NO:2), and nucleic acids comprising 1, 2, 3, 4, 5, 6, 7, 8, or more nucleotide exchanges, mutations or substitutions compared to a nucleic acid of defined sequence (e.g., SEQ ID NO:2).

Preferably, a variant of SEQ ID NOs: 1-12, 16, 17, 18, or 19 is capable of activating Toll-like receptor (TLR) 13. Alternatively and/or additionally, it is preferred that a variant of SEQ ID NOs: 1-12, 16, 17, 18, or 19 is capable of activating TLR13 expressing cells. Alternatively and/or additionally, a variant of SEQ ID NOs: 1-12, 16, 17, 18, or 19 is capable of stimulating an immune response in a non-primate subject, preferably in a non-human subject.

TLR13 activation or inhibition, respectively; or activation or inhibition of TLR13 expressing cells, respectively, is preferably done by bringing macrophages or conventional dendritic cells (cDCs), preferably TLR23479^{-/-} conventional dendritic cells (cDCs) or signal regulatory protein α (Sirp)^{high} cDCs in contact with a nucleic acid or variant of the invention and determining the amount of cytokines, preferably IFN-λ, IL-6, IL-12p70, and/or TNFα, and/or NO produced from said cells in comparison to such cells that were not brought into contact with such a nucleic acid or variant in case activation should be tested. If the amount of cytokines and/or NO increases in cells brought into contact with a suspected or known TLR13 activating nucleic acid or variant in comparison to a cell that was not brought into contact, then the potential TLR13 activating nucleic acid or variant is activating TLR13. As a positive control which will result in cytokine and/or NO production, for example, the nucleic acid shown in SEQ ID NO: 2 may serve.

In case, inhibition is tested, macrophages or conventional dendritic cells (cDCs), preferably TLR23479^{-/-} cDCs or signal regulatory protein α (Sirp)^{high} cDCs are stimulated with a nucleic acid or variant of the invention that activates TLR13. Afterwards, macrophages or conventional dendritic cells (cDCs), preferably TLR23479^{-/-} cDCs or signal regulatory protein α (Sirp)^{high} cDCs are brought into contact with a nucleic acid or variant thereof suspected or known to inhibit TLR13 activation and the amount of cytokines and/or NO is determined. If the amount of cytokines and/or NO decreases in cells brought into contact with a potential TLR13 inhibiting nucleic acid or variant in comparison to a cell that was stimulated before, but is not brought into contact with a potential TLR13 inhibiting nucleic acid or variant, then the potential TLR13 inhibiting nucleic acid or variant is inhibiting TLR13.

Preferably, as a control for the specificity of the test TLR23479^{-/-} pDCs that express TLR12 and lack TLR11, -13 may serve. These cells lack TLR-13 and, thus, are suitable to indicate as to whether any effect seen with TLR23479^{-/-} cDCs is specific for TLR-13, since TLR23479^{-/-} pDCs lack TLR-13 and thus, if they respond, then the nucleic acid or variant may not only be specific for TLR-13, but also for other TLRs, though this is not preferred.

Macrophages or conventional dendritic cells (cDCs), preferably TLR23479^{-/-} cDCs and pDCs or signal regulatory protein α (Sirp)^{high} cDCs may be obtained from mice lacking TLR2, 3, 4, 7, and 9. Such mice can be obtained by crossings. TLR23479^{-/-} cDCs also express CD8^{high} and TLR11, -12 and -13. The generation of cDCs and pDCs is described in documents 31 and 32.

As a negative control which will essentially not, preferably will not result in IL-6 and/or IL-12 production, for example, the nucleic acid shown in SEQ ID NO: 13

However, preferably, on the other hand a variant of SEQ ID NOs: 13, 14, or 15 is capable of inhibiting Toll-like receptor (TLR) 13. Alternatively and/or additionally, it is preferred that a variant of SEQ ID NOs: 13, 14, or 15 is capable of inhibiting TLR13 expressing cells. Alternatively and/or additionally, a variant of SEQ ID NOs: 13, 14, or 15 is capable of inhibiting an immune response in a non-primate subject, preferably in a non-human subject.

The term “immunostimulant” or “immunostimulator” or “immunostimulatory drug” or “immunostimulatory agent” means an agent that stimulates the immune system by inducing or increasing activity of any of its components. An immunostimulatory therapy may be considered for treating new-born or elderly subjects, or subjects otherwise having an immature immune system or impaired immune responses. An immunostimulatory therapy may also be indicated in the case of cancer.

The term “immunosuppressant” or “immunosuppressive drug” or “immunosuppressive agent” means an agent that inhibits or prevents activity of the immune system. An immunosuppressive therapy may be indicated in case of an excessive immune response, e.g. in septic syndrome, autoimmune diseases or allergies, or in order to prevent rejection of transplants.

The term “adjuvant” means an immunomodulatory agent, i.e. an immunostimulatory or immunosuppressive agent, which modifies the effect of other immunological agents. An adjuvant is often included in vaccines in order to enhance the recipient's immune response to the antigen. For example, an adjuvant enhancing the effect of another immunological agent may help keeping this immunological agent to a minimum.

### DETAILED DESCRIPTION OF THE INVENTION

Here we have identified an ssRNA segment within the peptidyl transferase loop of bacterial 23S ribosomal (r) RNA that binds antibiotics of the MLS group as a ligand of the orphan receptor TLR13. In particular, we have shown by gene knockdown, gain of function experiments, as well as specific RNase treatments and fractionation of total bacterial RNA that TLR13 recognises the bacterial 23S rRNA segment “ACGGAAAGACC” (SEQ ID NO: 2).

Working with mice and murine immune cells it was found that certain Gram-positive bacteria, such as Staphylococcus aureus or Streptococcus pneumoniae were seen by immune cells even if other TLRs, previously described to be involved in the recognition of Gram-positive bacteria (TLR23479^{-/-}), were excluded. The stimulatory agent within Gram-positive bacteria was found to be sensitive to RNase treatment suggesting that RNA was the stimulatory component. Using different dendritic cell subsets, known to selectively express different combinations of TLR11, TLR12 and TLR13 allowed the identification of TLR13 as the PRR for the stimulatory RNA of Gram-positive bacteria. The separation of total RNA of Gram-positive bacteria identified ribosomal RNA as the stimulatory component. Synthetic RNA oligonucleotides from the 23S rRNA of bacteria down 12 base pairs lengths were highly stimulatory to TLR13. Certain methylations and base substitutions of those oligos abrogated TLR13.

Thus, the invention describes for the first time molecular natural as well as artificial agonists for the activation of TLR13. This allows using these agonists for the activation of immune responses via TLR13. This allows further the design of antagonists to inhibit unwanted TLR13 activation. Some exemplary TLR13 activating and inhibiting nucleic acids are shown in Table 1:

TABLE 1
S. aureus 23S rRNA mimicking and derived oligoribonucleotides (ORNs)

| Name | sequence | TLR7* | TLR13** |
|---|---|---|---|
| SaI: | 5′-CCGACACAGGUAGUCAAGAU-3′ | n.a. | - |
| | (SEQ ID NO: 33) | | |
| SaII: | GCACCUCGAψGUCGC (ψ, pseudouridine) | n.a. | - |
| | (SEQ ID NO: 34) | | |
| SaIIIas: | 3′-CCAAUGGGCGCUGUCCUGCCUUUCUGGGGCACCUCGAAAUGACAUCGG-5′ | - | ++ |
| | (SEQ ID NO: 15) | | |
| SaIII: | 5′-GGUUACCCGCGACAGGACGGAAAGACCCCGUGGAGCUUUACUGUAGCC-3′ | ++ | +++ |
| | (SEQ ID NO: 11) | | |
| SaIIId3: | GGUUACCCGCGACAGGACGGAAAGACCCCGUG | - | +++ |
| | (SEQ ID NO: 4) | | |
| SaIIId5: | GACGGAAAGACCCCGUGGAGCUUUACUGUAGCC | ++ | +++ |
| | (SEQ ID NO: 12) | | |
| Sa23: | CAGGACGG [symbol] AAGACCCCGUGGAG | - | +++ |
| | (SEQ ID NO: 5) | | |
| Sa19: | GGACGGAAAGACCCCGUGG | - | +++ |
| | (SEQ ID NO: 6) | | |
| Sa17: | GACGGAAAGACCCCGUG | - | +++ |
| | (SEQ ID NO: 7) | | |
| Sa12: | GACGGAAAGACC | - | +++ |
| | (SEQ ID NO: 8) | | |
| Sa9: | GGAAAGACC | - | - |
| | (SEQ ID NO: 20) | | |
| Sa12s7: | GACGGACAGACC | - | (+) |
| | (SEQ ID NO: 21) | | |
| Sa12s6: | GACGG [symbol] AAGACC | - | - |
| | (SEQ ID NO: 22) | | |
| Sa12s5: | GACGAAAAGACC | - | (+) |
| | (SEQ ID NO: 23) | | |
| Sa1l2st: | AAAAGAAAGAAA | - | - |
| | (SEQ ID NO: 24) | | |
| Sa12t3a: | GACGGAAAGAAA | - | + |
| | (SEQ ID NO: 25) | | |
| Sa12t5a: | AAAAGAAAGACC | - | (+) |
| | (SEQ ID NO: 26) | | |
| Sa12t3b: | GACGGAAAGACA | - | + |
| | (SEQ ID NO: 27) | | |
| Sa12t5b: | AACGGAAAGACC | - | +++ |
| | (SEQ ID NO: 3) | | |
| Sa12t5c: | AAAGGAAAGACC | - | + |
| | (SEQ ID NO: 28) | | |
| Sa12sec: | GACCCAAAGAGG | - | - |
| | (SEQ ID NO: 29) | | |
| | m | - | - |
| Sa12m6: | GACGG [symbol] AAGACC | | |
| | m | | |
| | (SEQ ID NO: 30) | | |
| | mm | - | ++ |
| Sa12m7: | GACGG [symbol] AAGACC | | |
| | (SEQ ID NO: 31) | | |
| Sa12mm: | GACGG [symbol] AAGACC | - | - |
| | (SEQ ID NO: 32) | | |
| Sa12A19: | AAACGGAAAGACCAAAAAA | - | +++ |
| | (SEQ ID NO: 9) | | |
| Sa19PSO: | GGACGGAAAGACCCCGUGG | - | +++ |
| | (SEQ ID NO: 10) | | |

*IFN from wt-FL-pDCs upon challenge with 100 pmol ORN (+Lyovec) per well
**IL-6 [pg/ml] from total wt-BM cells upon challenge with 100 pmol ORN + LV/well; -, <100; (+), 100-199, +, 200-499; ++, 500-1999; +++, >2000; n.a., not analyzed; bold, position of A that it methylated within total 23S rRNA; italics, mutated or methylated; PSO/underlined, stabilized by thioate modification.

In more detail, a TLR13 activating minimal nucleic acid sequence is as follows:

SEQ ID NO: 1
(ACGGAAXXXCC),
or
SEQ ID NO: 2
(ACGGAAAGACC)

Nucleic acid sequences found to activate TLR13 are as shown in Table 1:
SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), or SEQ ID NO: 10 (Sa19PSO)

Nucleic acid sequences found to activate both TLR13 and TLR7 are as shown in Table 1:
SEQ ID NO: 11 (SaIII) or SEQ ID NO: 12 (SaIIId5)

A TLR13 inhibiting nucleic acid is as follows:

SEQ ID NO: 13
(UGCCUUXXXGG),
or
SEQ ID NO: 14
(UGCCUUUCUGG)

A TLR13 inhibiting nucleic acid found to inhibit TLR13 is as shown in Table 1:
SEQ ID NO: 15 (SaIIIas).

Further TLR13 activating nucleic acid sequences are as follows:

SEQ ID NO: 16
(GCCGGAAAGACC; Sa12s2),
SEQ ID NO: 17
(GACGGAACGACC; Sa12s8),
SEQ ID NO: 18
(GACGGAAAAACC; Sa12s9),
or
SEQ ID NO: 19
(GACGGAAAGCCC; Sa12s10)

A nucleic acid of the present invention is preferably in an ‘isolated’ form. The term “isolated” as used herein in the context of a nucleic acid refers to removal of the nucleic acid from its natural source, environment or milieu.

The present invention may also be characterized by the following items:

1) A nucleic acid comprising or consisting of a sequence of SEQ ID NO: 1 (ACGGAAXXXCC) or a variant thereof for use as a pharmaceutical agent.
2) The nucleic acid according to item 1, comprising or consisting of a sequence of SEQ ID NO: 2 (ACGGAAAGACC) or a variant thereof.
3) The nucleic acid according to item 1 or 2, comprising or consisting of a sequence selected from the group consisting of SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO) or a variant thereof.
4) The nucleic acid according to item 3, comprising or consisting of a sequence of SEQ ID NO: 10 (Sa19PSO) or a variant thereof.
5) The nucleic acid according to item 1 or 2, comprising or consisting of SEQ ID NO: 11 (SaIII) or SEQ ID NO: 12 (SaIIId5) or a variant thereof.
6) The nucleic acid according to any of the preceding items for use as a pharmaceutical agent for activating Toll-like receptor (TLR) 13 expressing cells in a subject.
7) The nucleic acid according to any of the preceding items, wherein the pharmaceutical agent is an immunostimulant.
8) The nucleic acid according to any of the preceding items, wherein the pharmaceutical agent is an adjuvant, preferably for vaccination against bacterial infection.
9) The nucleic acid according to any of the preceding items for use as a pharmaceutical agent for treating an infection by a Gram-positive or Gram-negative bacterium resistant to one or more antibiotics, preferably resistant to one or more antibiotics of the macrolide, lincosamide, and streptogramin (MLS) group, most preferably resistant to erythromycin.
10) The nucleic acid according to item 9, wherein the Gram-positive bacterium is Staphylococcus aureus or Streptococcus pneumoniae.
11) A pharmaceutical composition comprising a nucleic acid comprising or consisting of a sequence of SEQ ID NO: 1 (ACGGAAXXXCC) or SEQ ID NO: 2 (ACGGAAAGACC) or a variant thereof.
12) The pharmaceutical composition according to item 11, comprising a nucleic acid comprising or consisting of a sequence selected from the group consisting of SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), SEQ ID NO: 10 (Sa19PSO), SEQ ID NO: 11 (SaIII), and SEQ ID NO: 12 (SaIIId5) or a variant thereof, preferably comprising or consisting of a sequence of SEQ ID NO: 10 (Sa19PSO) or a variant thereof.
13) A nucleic acid comprising or consisting of a sequence of SEQ ID NO: 13 (UGCCUUXXXGG) or a variant thereof for use as a pharmaceutical agent.
14) The nucleic acid comprising or consisting of a sequence of SEQ ID NO: 14 (UGCCUUUCUGG) or a variant.
15) The nucleic acid according to item 13 or 14, comprising or consisting of a sequence of SEQ ID NO: 15 (SaIIIas) or a variant thereof.
16) The nucleic acid according to any of items 13 to 15 for use as a pharmaceutical agent for inhibiting TLR13 expressing cells in a subject.
17) The nucleic acid according to any of items 13 to 16, wherein the pharmaceutical agent is an immunosuppressant.
18) The nucleic acid according to any of items 13 to 17, for use as a pharmaceutical agent for treating septic syndrome induced by a Gram-positive or Gram-negative bacterium.
19) The nucleic acid according to item 18, wherein the Gram-positive bacterium is Staphylococcus aureus or Streptococcus pneumoniae.
20) A pharmaceutical composition comprising a nucleic acid comprising or consisting of a sequence of SEQ ID NO: 13 (UGCCUUXXXGG) or SEQ ID NO: 14 (UGCCUUUCUGG) or a variant thereof.
21) The pharmaceutical composition according to item 20, comprising a nucleic acid comprising or consisting of a sequence of SEQ ID NO: 15 (SaIIIas) or a variant thereof.
22) An in vitro method for activating TLR13 expressing cells, or for inducing cytokine and/or NO release from TLR13 expressing cells, comprising the step of contacting the cells with a nucleic acid comprising or consisting of a sequence of SEQ ID NO: 1 (ACGGAAXXXCC) or a variant thereof.
23) An in vitro method for studying TLR13 mediated cell activation, comprising the steps of:
a) contacting TLR13 expressing cells, or cells to be examined for TLR13 expression, with a nucleic acid comprising or consisting of a sequence of SEQ ID NO: 1 (ACGGAAXXXCC) or a variant thereof;
b) determining cytokine and/or NO release from the cells.
   24) The in vitro method according to item 23, further comprising the step of contacting the cells with an inhibitor of TLR13 mediated cell activation prior to step (a), preferably with an antisense oligonucleotide comprising or consisting of a sequence of SEQ ID NO:
   13 (UGCCUUXXXGG) or SEQ ID NO: 14 (UGCCUUUCUGG) or a variant thereof, most preferably comprising or consisting of a sequence of SEQ ID NO: 15 (SaIIIas) or a variant thereof.
   25) The in vitro method according to any of items 22 to 24, wherein the nucleic acid comprises or consists of a sequence selected from the group consisting of SEQ ID NO: 2 (ACGGAAAGACC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO) or a variant thereof.
   26) The in vitro method according to any of items 22 or 25, wherein the nucleic acid comprises or consists of SEQ ID NO: 11 (SaIII) or SEQ ID NO: 12 (SaIIId5) or a variant thereof.
   27) The in vitro method according to any of items 22 to 26, wherein the cytokine is selected from the group consisting of IFN-λ, IL-6, IL-12p70, and TNFα.
   28) The in vitro method according to any of items 22 to 26, wherein the TLR13 expressing cells are macrophages or conventional dendritic cells (cDCs).
   29) The in vitro method according to item 28, wherein the cDCs are TLR23479^{-/-} eCD8^{high} cDCs or signal regulatory protein α (Sirp)^{high} cDCs.
   30) Use of a nucleic acid comprising or consisting of a sequence selected from the group consisting of a sequence of SEQ ID NO: 1 (ACGGAAXXXCC), SEQ ID NO: 2 (ACGGAAAGACC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO), SEQ ID NO: 11 (SaIII), SEQ ID NO: 12 (SaIIId5), SEQ ID NO: 13 (UGCCUUXXXGG), SEQ ID NO: 14 (UGCCUUUCUGG), and SEQ ID NO: 15 (SaIIIas) or a variant thereof for studying TLR13 expressing cells or TLR13 mediated signal transduction.
   31) A nucleic acid comprising or consisting of a sequence selected from the group consisting of SEQ ID NO: 1 (ACGGAAXXXCC), SEQ ID NO: 3 (Sa12t5b), SEQ ID NO: 4 (SaIIId3), SEQ ID NO: 5 (Sa23), SEQ ID NO: 6 (Sa19), SEQ ID NO: 7 (Sa17), SEQ ID NO: 8 (Sa12), SEQ ID NO: 9 (Sa12A19), and SEQ ID NO: 10 (Sa19PSO), SEQ ID NO: 11 (SaIII), SEQ ID NO: 12 (SaIIId5), SEQ ID NO: 13 (UGCCUUXXXGG), SEQ ID NO: 14 (UGCCUUUCUGG), and SEQ ID NO: 15 (SaIIIas) or a variant thereof.

In the following, the present invention will be described in more detail on the basis of the examples and with reference to the accompanying figures, in which

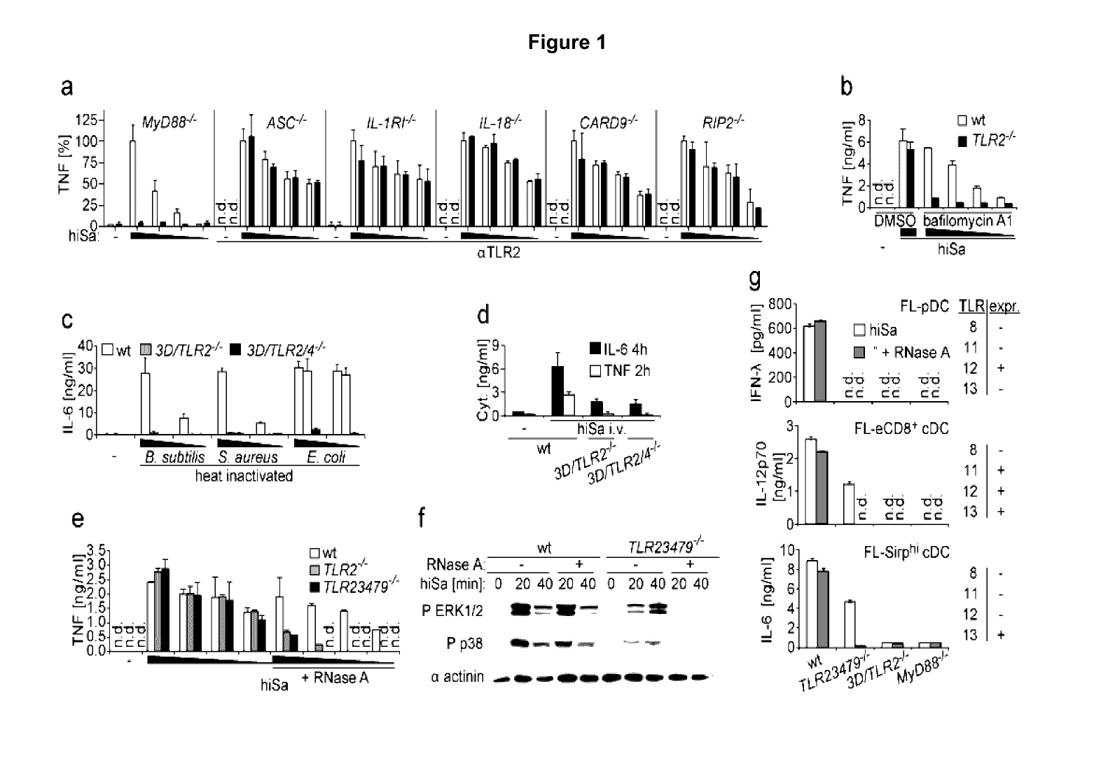

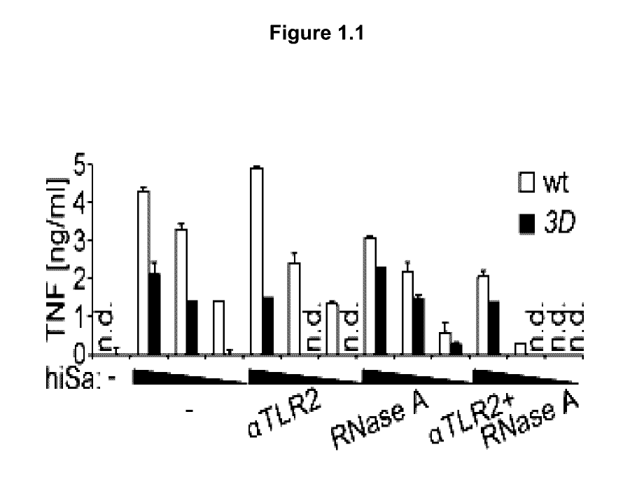

FIG. 1 (a-q) shows that Gram-positive bacteria activate TLR23479^{-/-} macrophages and DCs via MyD88. Macrophages were preincubated in 20 μg/ml of a TLR2 neutralizing antibody (if indicated) and challenged with 10⁹ (rectangle) and 10⁸, as well as 10⁷ and 10⁶ cfu/ml (indicated by triangles) of heat inactivated S. aureus (hiSa, titered prior to inactivation). Culture supernatants were sampled and analyzed by ELISA. a, Results of wt macrophages challenge with 10⁹ cfu/ml hiSa was set to 100% (representing 2 to 10 ng/ml) to which the other concentrations within the respective block were related (challenge for 8 h). b, Macrophages were preincubated for 30 min with dimethyl sulfoxide (DMSO) alone or with 50 nM bafilomycin A before 8 h microbial challenge. c, Macrophages were challenged for 16 h. d, Mice were challenged intravenously (i. v.) with 10⁹ cfu hiSa. Serum samples were analyzed by ELISA (the data are representative for 3 experiments, each group with n=3 mice). e-g, The indicated hiSa suspensions have been preincubated with RNase A (+) prior to challenge of macrophages (e, f) or DCs (g) for 16 h (e, g) or times indicated (f, lysates were subjected to SDS PAGE and immunoblot analysis); P, phosphorylated. g, Flt3-ligand (FL)-derived DC subsets were challenged for 16 h and the cytokine contents of the supernatants were analyzed. The respective TLR expression (expr.) in DC subset equivalents is indicated (hi, high; −, no detectable expression; +, expression).

FIG. 1.1 shows that Gram-positive bacteria activate 3D macrophages unless TLR2 is blocked. Macrophages were pre-incubated with 20 μg/ml of a neutralizing antibody (α) towards TLR2 if indicated and were challenged with 10⁹, 10⁸, and 10⁷ cfu/ml heat inactivated S. aureus (hiSa, triangles), which was either untreated or pre-treated with RNaseA. Supernatants were sampled 16 h later and analyzed by ELISA.

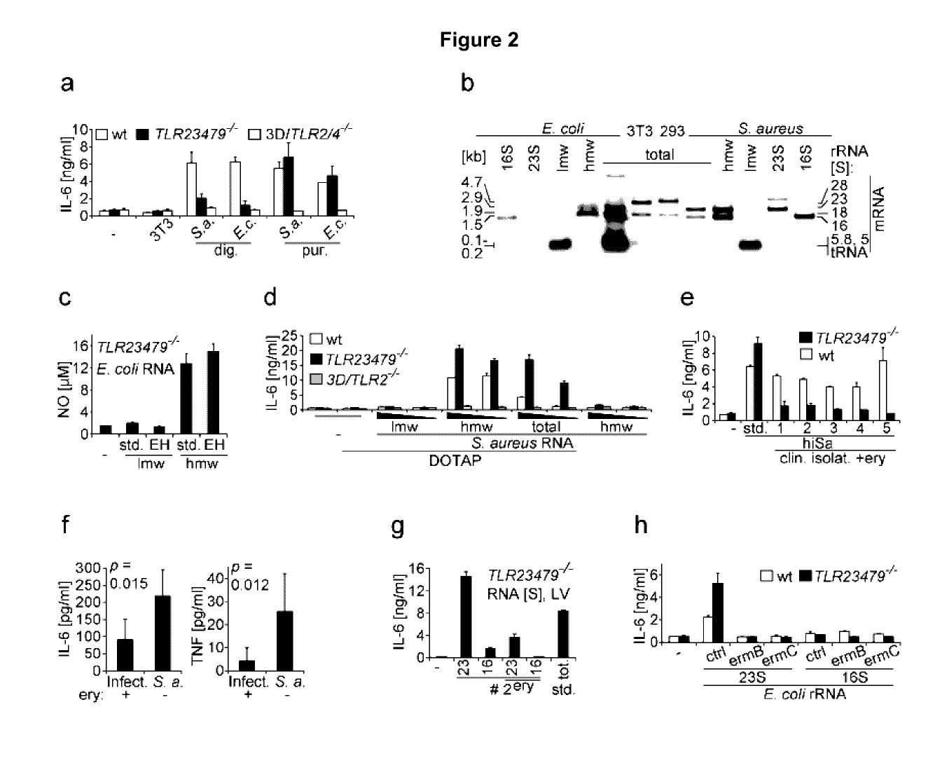

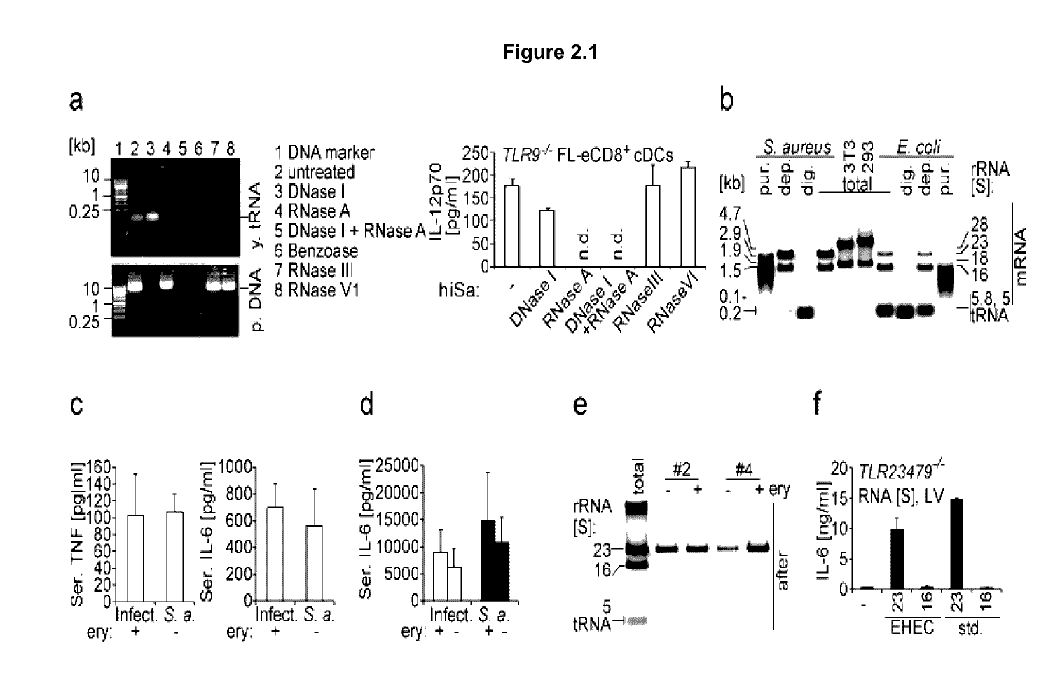

FIG. 2 (a-h) shows that bacterial 23S rRNA is stimulatory unless its A2085/2058 is methylated. In a, c, d, e, g, h, After challenge of macrophages for 16 h, supernatants were sampled for cytokine or nitrite (NO) content. a, Bacterial RNA preparations resulting from incubation of total RNAs with 5′-phosphate-specific exo RNase targeting large rRNAs (dig.) or precipitation of both large rRNAs (pur.) were applied to macrophages. b, Bacterial RNA together with eukaryotic cell line RNA preparations were gel-electrophoresed analytically (kb, kilo bases; S, Svedberg; r, ribosomal; t, transfer; m, messenger). b-d, Bacterial total RNAs were separated by anion-exchange chromatography into low molecular weight (lmw) and high molecular weight (hmw) fractions, or by agarose gel electrophoresis after which 16S and 23S rRNA-containing gel slices were cut out and RNA was re-isolated and subsequently transfected into macrophages (std., regular E. coli isolate; EH, enterohemorrhagic E. coli). e, Erythromycin (ery) resistant clinical S. aureus isolates (clin. isolat.) were cultured in 10 mg/l erythromycin (+ery). Macrophages were challenged with 10⁹ cfu/ml heat inactivated erythtromycin sensitive (std.) or -resistant (numbered from 1 to 5) S. aureus (hiSa). f, TLR23479^{-/-} mice (n=6) were infected (Infect.) intravenously with logarithmically growing 10⁸ cfu erythromycin resistant S. aureus (S. a.) clinical isolate that had been grown in the presence (+) or absence (−) of erythromycin. Serum was drawn after 2 h and analyzed for cytokines by cytometric bead array. g, Next, 23S (23) or 16S (16) rRNA, or total (tot.) RNA preparations from numbered clinical isolate (#2) or control (std.) S. aureus grown in the presence of erythromycin (ery) or in its absence were transfected with lyovec (LV). h, E. coli BL21 was transformed with erythromycin resistance methyltransferase (erm) B or C expression plasmids or not (ctrl, control) and cultured. Large rRNAs were isolated and transfected into macrophages.

FIG. 2.1 (a-f) shows that single-stranded bacterial 23S RNA activates TLR23479 macrophages.

a, Yeast (y.) tRNA and plasmid (p.) DNA were treated with the indicated nucleases as control and applied to an agarose gel. Heat-inactivated S. aureus (hiSa) bacteria were nuclease treated as indicated and FL-DCs were challenged with 10⁷ cfu/ml nuclease- or PBS-treated (−) hiSa for 16 h. b, Total bacterial RNAs were incubated with 5′-phosphate-specific exo RNase targeting large rRNAs (dig.), 5′phosphate-specific phosphatase (dep.), or subjected to precipitation of both large rRNAs (pur.; kb, kilo bases; S, Svedberg; r, ribosomal; t, transfer; m, messenger). c, wt mice (n=6) were infected with 10⁸ cfu logarithmically growing clinical erythromycin-resistant S. aureus (S. a.) isolate cultured in the presence (+) or absence (−) of erythromycin (ery). Serum was drawn 2 h after infection and analyzed by CBA. d, Infection was performed as described in b 16 h upon which serum was drawn (white columns, wt; black columns, TLR23479^{-/-}; n=6 for each genotype). e, Agarose gels carrying isolated 23S rRNA fractions isolated from total RNA of resistant S. aureus (#, number of isolate) grown in the presence (+) or absence (−) of erythromycin (ery). f, Macrophages were transfected with Lyovec (LV) for 16 h with large rRNA fractions from entero-hemorrhagic E. coli (EHEC) or regular clinical isolate (std.) E. coli.

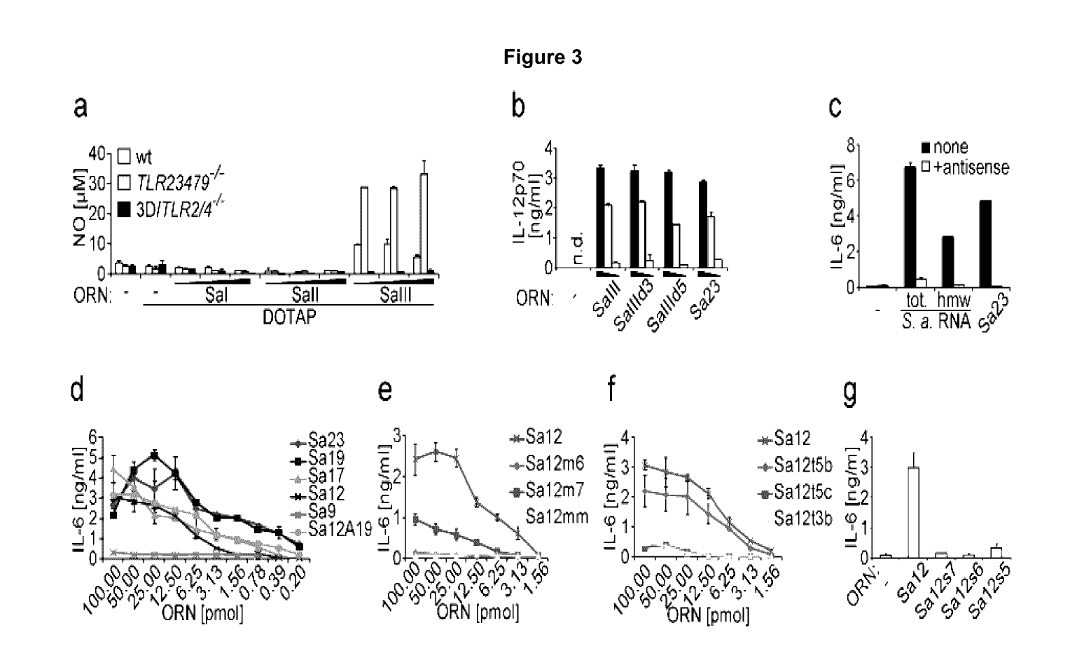

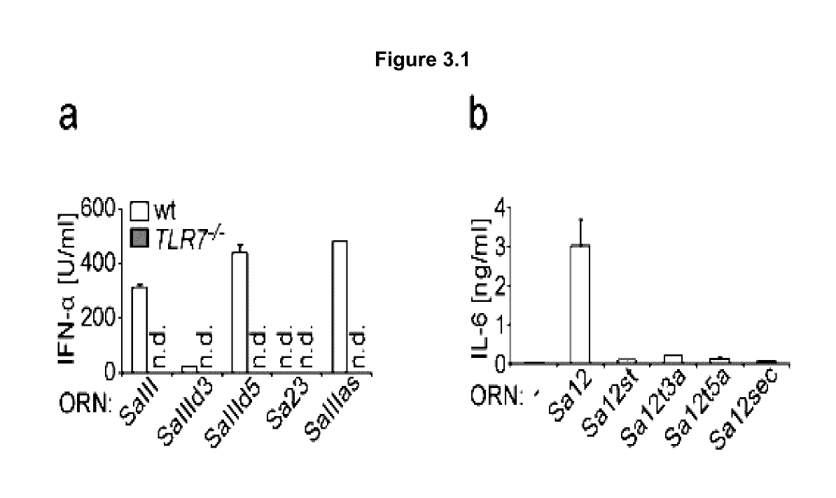

FIG. 3 (a-q) shows that ORNs harbouring the 23S rRNA sequence around A2085/2058 activate macrophages and cDCs.

a-g, Cells were challenged for 16 h. Thereafter supernatants were assayed for proinflammatory cytokine contents. a, Sequence motifs covering 3 separate methylation sites in S. aureus 23S rRNA were mirrored by ORNs (see sequences in Table 1), which were transfected into macrophages. b, TLR23479 FL-eCD8⁺ cDCs were transfected with the ORNs indicated (amount/well [pmol]: black, 10; grey, 1; white, 0.1). c, TLR23479^{-/-} Sirp^{high} cDCs were transfected with the S. aureus RNA preparations indicated or an ORN covering the SaIII core sequence (10 pmol/well), either in the absence (none) of or upon preincubation for 20 min with 100 pmol/well antisense RNA ORN (SaIIIas, +antisense). d,-g, Bone marrow cells were challenged with transfected ORNs at doses indicated (or 100 pmol/well if not indicated).

FIG. 3.1 (a-b) shows that TLR13-activating ORNs additionally activate TLR7, and specific mutations abrogate TLR13 activation.

a, b, FL-pDCs of the genotypes indicated (a) and wt bone marrow cells (b) were challenged with 100 pmol/well ORNs by transfection (Lyovec) for 16 h.

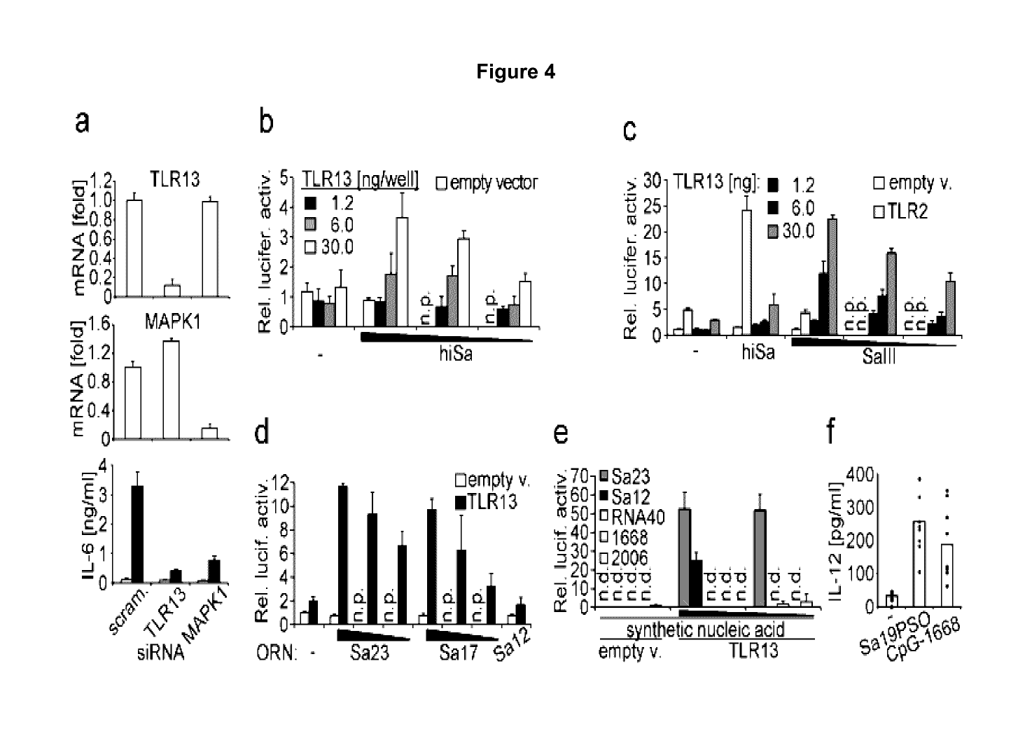

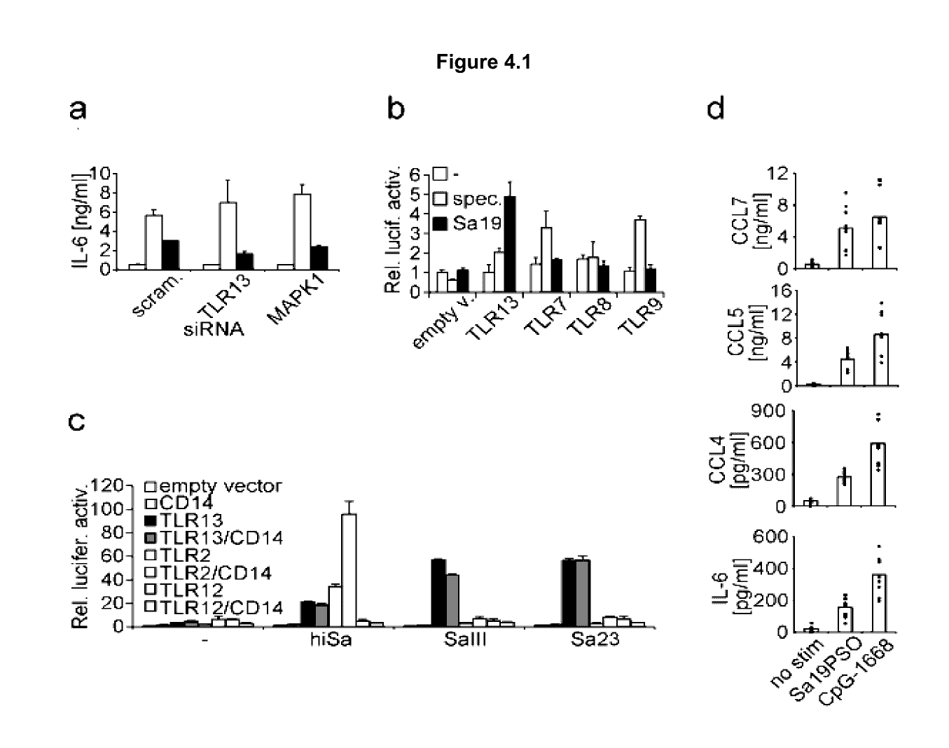

FIG. 4 (a-f) shows that TLR13 recognises heat-inactivated S. aureus and ORNs mirroring bacterial 23S rRNA segments covering A2085/2058.

a, Macrophages were transfected with siRNAs to knock down specific mRNA accumulation (MAPK1, ERK2) or with control siRNA (scram., upper diagrams). Upon transfection cells were seeded and left untreated (white columns in lower diagram), or challenged with 100 pmol/well ORN SaIII (black columns) for 16 h. Supernatants were analyzed by ELISA. b-e, Human embryonic kidney (HEK) 293 line cells were transfected with TLR expression and luciferase reporter plasmids. After 24 h they were challenged for 16 h to analyze NF-κB driven relative (rel.) luciferase (lucifer. or lucif.) activity (activ.); n.d., not detectable; n.p., not performed; v., vector; −, no challenge. b, c, With varying doses or one dose of TLR expression plasmid (TLR2: 2 ng) or empty (30 ng) vector transfected cells were challenged with 10⁹ (no indication, c) or additionally 10⁸ and 10⁷ cfu/ml of heat-inactivated S. aureus (hiSa, triangle, b), or 100 (d, e) or 10, or 1 pmol/well ODN (triangle, c-e). d, e, 15 ng/well TLR13 expression plasmid was transfected. e, Either 10 μM of CpG-DNA only, or additionally 1 μM of CpG-DNA (triangle) was applied. ORNs and CpG-DNA were transfected with DOTAP. f, Next, 10 nmol of ORN or CpG-DNA were given i. v. into mice (n=9). Serum was drawn 6 h later from challenged and untreated mice (−) for analysis by cytometric bead arrays (IL-12, IL-12p70).

FIG. 4.1 (a-d) shows that specifically TLR13 mediates cellular and systemic recognition of the 23S rRNA segment that encompasses A2085/2058.

a, Macrophages were transfected with siRNAs specific for the mRNA molecules indicated (MAPK1, ERK2) or control siRNA (scram.). After transfection, cells were seeded and left untreated (white columns) or challenged for 16 h with 10⁸ or 10⁷ cfu/ml heat inactivated S. aureus (hiSa; light grey or dark grey columns, respectively). b, c, Human embryonic kidney (HEK) 293 cells were transfected for 24 h with plasmids for expression of pattern recognition receptors (PRRs) as indicated or with empty vector (v.) and NF-κB reporter plasmid upon which cells were challenged for 16 h and lysed to analyze relative (rel.) luciferase (lucifer. or lucif.) activity (activ.). b, Cells were left untreated (−), challenged with individual stimuli (spec.; TLR13: 10⁸ cfu/ml hiSa; TLR7: 4 μg/ml CL075; TLR8: 10⁸ cfu/ml hiSa; TLR9: 2 μM CpG-1668) or transfected with 100 pmol/well Sa10 by Lyovec. c, Cells were left untreated (−) or challenged with 10⁸ cfu/ml hiSa or 100 pmol/well of ORNs indicated. d, Mice were challenged by i. v. injection of 10 nmol of ORN or CpG-DNA (n=9). Serum was drawn 6 h later from treated and untreated (no stim) mice for analysis of cytokine content by cytometric bead array.

### EXAMPLES

Materials and Methods

Materials. RNase free DNase I (Roche), RNaseH (Fermentas), DNase I and RNaseA (Sigma), RNaseIII (NEB), and RNaseVI (Ambion) were applied according to supplier indications. Cells were preincubated with anti TLR2 antibody⁹ (clone T2.5, HBT) and bafilomycin A (Sigma) used as blockers. Anti phospho-ERK1/2 and -p38 (cell signaling) and anti alpha actinin (Santa Cruz) antibodies were used for immuno blot analysis. ORNs (IBA or Metabion) as well as CpG DNA-1668 and -2006 (TIB-Molbiol) were in aqueous solutions. Dotap (Roche) and lyovec (Invivogen) were used for transfections.

Bacteria. S. aureus (DSMZ 20231), B. subtilis (DSMZ 10), and S. pneumoniae (D39), as well as clinical isolates of E. coli⁹ including EHEC O104:H4 and S. aureus were used for challenges in vivo or in vitro, or as sources of RNA preparations used for cell transfection (1 μg total RNA or 0.2 μg RNA fraction per well of a 96 well plate using lyovec normally). For bacteria preparation and erythromycin conditioning, bacteria were cultured in the absence or presence of 10 μg/ml erythromycin and were inoculated with 16 h grown preparatory cultures (agitation, 37° C., E. coli in LB medium, S. aureus, B. subtilis, and S. pneumoniae in BHI, the latter in CO₂ incubator). In the logarithmic phases of main cultures, which were delayed by erythromycin for 40 min, bacteria were pelleted and collected in PBS for RNA preparation, heat inactivation (incubation in boiling water for 15 min), or infection. Bacterial samples (taken prior to inactivation) were always titered⁹.

Mice. CARD9^{-/-}, RIP2^{-/-}, ASC^{-/-}, IL-1R1^{-/-}, and IL-18^{-/-} mice were bred at the animal facility of the Institute of Medical Microbiology, Immunology, and Hygiene, Technical University of Munich. Wt C57BL/6, TLR23479 and single TLR k. o. mice¹⁵, 3D¹³ (acquired from MMRC of the Scripps Research Institute) from which 3D/TLR2^{-/-} and 3D/TLR2/4^{-/-} were derived by cross breeding, as well as MyD88^{-/-1} mice were used as sources of in vitro generated DC subpopulations and macrophages. Challenged mice were anaesthetized by short incubation in an isoflurane saturated glass barrel. Thereupon blood was sampled by retrobulbar dotting. Animal experiments were approved by local authorities.

Cells and challenges. Macrophages and DCs were derived from bone marrow cells while HEK293 cells were grown as cell line^{26,27}. FL-DCs were generated, FACS sorted, stimulated in the presence of IL-12p70 promoting cytokines, and challenged. Thereafter supernatant cytokine amounts were determined²⁷. Total bone marrow cells were transfected with ORNs. Thereafter IL-6 was analysed by ELISA.

RNA preparations. Bacterial RNAs were prepared by acidic phenol extraction²⁸. Briefly, bacteria were washed and solubilised in 50 nM sodium azide, raised in glass milk (ribolyzer, MPbio), extracted, and precipitated. 1% agarose gels using MOPS running buffer were applied for RNA analysis and fractionation. The 16S and 23S rRNAs were dephosphorylated with RNA 5′-polyphosphatase and digested with terminator 5′-phosphate-dependent exonuclease (both from Epicentre Biotechnologies). The Microbeexpress bacterial mRNA enrichment kit (Ambion) including 16/23S rRNA complementary DNA coupled to magnetic beads was used to purify both of the two large rRNAs together. Lmw and hmw fractions were isolated from total RNA by anion-exchange chromatography using nucleobond RNA/DNA 400 columns (Macherey-Nagel) while total RNA was separated on a preparative agarose gel from which slices were cut out that contained 16S or 23S rRNA which subsequently were purified (Zymoclean gel RNA recovery kit, Zymoresearch). For RT PCR analysis of mRNA expression macrophages were opened up by 2 min panning in phenol solution (trifast, Peqlab). Upon addition of chloroform (Roth) the aqueous was separated from the organic phase by centrifugation and RNA precipitated for 16 h at 4° C., washed in ethanol and resuspended in water by incubation at 55° C. for 10 min.

Cytokine and NO measurement. ELISA (R&D) and CBA (e-bioscience) was as described^{9,27}. Nitrite was quantified according to the Griess protocol to indicate cellular NO release. Briefly, 0.2% N-(1-naphtyl) ethylene diamine dihydrochloride was mixed with 2% sulphanilamide in 5% phosphoric acid (all Sigma) as 1:1 (by volume). This mixture was mixed with cell culture supernatant as 1:1 (by volume). Resultant absorption at 540 nm was determined immediately to calculate nitrite concentration by comparison to a sodium nitrite (Sigma) standard reference curve.

Phosphorylation analysis. Lysate analysis by immuno blotting was as described⁹.

Erm overexpression. Total RNA was isolated from a clinical erythromycin-resistant S. aureus isolate growing in 10 μg/ml erythromycin (logarithmic phase). The ermC cds was amplified by RT PCR and subcloned (sequenced). The ermB cds was subcloned from a Gram-positive bacteria expression vector pat18²⁹ (also used to exchange ermB cds by that of ermC to both transform into B. subtilis). Both cdss were ligated into the Gram-negative bacteria expression vector pGEX2T (GE lifesciences). Constructs were transformed into E. coli strain BL21 codon plus (Stratagene) for conference of erythromycin resistance.

mRNA knock down. Macrophages were generated by incubation of bone marrow cells in teflon foil bags (Sarstedt No. 94.6077.317)³⁰. siRNA (Qiagen) towards murine TLR13 mRNA (ID S101449518) and MAPK1/ERK2 mRNA (ID 1022564, positive control), as well as a scrambled variant (ID 1027310, negative control) were transfected by electroporation (gene pulser Biorad, exponential protocol, 400 V, 150 ρF, 100Ω) in optimem medium (Invitrogen). 48 h later cells were challenged for 16 h. Supernatants were analyzed by ELISA and Griess assay and RNA was isolated from cells (s. above). RNA was DNase I (Roche) digested, oligo dT18 primed and reversely transcribed (M-MuLv, Fermentas). DNA amplification by RT PCR (maxima sybr green/rox, Fermentas) was monitored (7500 Fast, Applied Biosystems). Threshold cycle (CT) was determined for each sample and related to respective actin sample value. Results for treated were related to those of respective untreated samples to calculate fold mRNA expression (2-ΔΔCT).

Luciferase assay. Murine TLR12 and TLR13 (Invivogen) or other PRR expression plasmids were transfected together with luciferase reporter plasmids into HEK293 cells for analysis of NF-κB driven and constitutive luciferase activities, as previously described²⁶.

Statistics. Students t-test for unconnected samples was applied to calculate significances.

### Example 1: Identification of TLR13 as a Single-Stranded (SS) RNA Sensor

We compared macrophages lacking the expression of caspase recruitment domain (CARD) 9 (CARD9^{-/-}), receptor-interacting protein 2 (RIP2^{-/-}), apoptosis-associated speck-like protein containing a CARD (ASC^{-/-}), IL-1 receptor 1 (IL-1R^{-/-}), IL-18 (IL-18^{-/-}), or MyD88 (MyD88^{-/-}) in terms of their responsiveness to heat-inactivated S. aureus (hiSa) or S. pneumoniae in the presence of a TLR2-blocking antibody (αTLR2)^{1,8-12}. Cytokine production was found to be strictly dependent on MyD88 (FIG. 1a), a finding that implies the involvement of MyD88-dependent TLRs.

Next we asked whether endosomal TLRs (TLR3, -7, -8, -9, -11, and -13) are involved in cell activation. We inhibited endosomal acidification with bafilomycin and analyzed UNC93B1-defective (3D) macrophages that lack ER-endosome TLR trafficking^{1,13,14}. Blocking of endosomal acidification abrogated recognition of Gram-positive bacteria in TLR2^{-/-} macrophages (FIG. 1b). Furthermore, 3D macrophages (or mice) lacking additionally TLR2 and -4 were unresponsive to bacterial challenge (FIG. 1c, d; FIG. 1.1). However, TLR23479^{-/-} macrophages or mice¹⁵ responded well to hiSa challenge unless the bacterial preparations were subjected to RNaseA treatment (FIG. 1e, f). Thus, hiSa was recognized by immune cells even if other TLRs, previously described to be involved in the recognition of Gram-positive bacteria (TLR23479^{-/-}), were excluded.

Dendritic cell (DC) subsets express different sets of TLRs¹⁶. We generated equivalents (e) of in vivo conventional (c) DC (known to express TLR13) and plasmacytoid (p) DC (lacking TLR13 expression) in vitro with flt3-ligand (FL). The responsiveness of these cells to hiSa was dependent on MyD88 and 3D. Specifically, TLR23479^{-/-} eCD8^{high} and signal regulatory protein α (Sirp)^{high} cDCs responded to hiSa, whereas TLR23479^{-/-} pDCs failed to do so (FIG. 1g).

Together, these findings indicate that TLR13 may be a bacterial single-stranded (ss) RNA sensor.

### Example 2: Identification of 23S rRNA as the Stimulatory ssRNA

To identify the relevant RNA, we incubated hiSa with DNAse I, calf intestinal phosphatase, 5″-phosphate-specific phosphatase (to affect the integrity of 16S and 23S rRNA), or double-stranded (ds) RNA-specific RNase III or VI. These treatments did not alter the stimulatory activity of hiSa, in line with a recent report (FIG. 2.1a, b)¹⁸. However, nucleic acid-degrading benzonase and ssRNA-specific RNaseA abrogated the TLR23479^{-/-} cDC and macrophage stimulatory activity of hiSa (FIG. 1e, f, g; FIG. 2.1a).

We then treated total RNA with 5″-phosphate-dependent exonuclease and precipitated large rRNAs (FIG. 2.1b) to narrow down the stimulatory activity. After transfection, 16S/23S rRNA isolates of both S. aureus and E. coli triggered the activation of TLR23479^{-/-} macrophages and cDCs while 16S/23S rRNA digestion abrogated stimulatory activity (FIG. 2a). Whereas low molecular weight (lmw) portions from total RNA lacked a stimulatory activity, high molecular weight (hmw) portions of Gram-negative and Gram-positive bacterial RNA activated TLR23479^{-/-} cells (FIG. 2b-d). These findings suggested that a fraction of large bacterial rRNAs activates macrophages and cDCs in a MyD88-dependent manner.

Because modification of bacterial rRNA, such as by the methyltransferases ermB or ermC, confers resistance to antibiotics such as erythromycin^{7,19}, we analyzed five clinical S. aureus isolates displaying various resistance phenotypes, including erythromycin resistance. When these isolates were grown in the presence of erythromycin, they largely lacked the capacity to immediately activate TLR23479^{-/-} mice and pure macrophages, whereas wild-type (wt) control responses were normal (FIG. 2e, f; FIG. 2.1c, d). In contrast, all erythromycin-resistant S. aureus isolates grown in the absence of erythromycin strongly stimulated TLR23479^{-/-} mice and cells (FIG. 2f). These results suggested an erythromycin-driven RNA camouflage from its receptor.

Accordingly and also in line with erythromycin-mediated N6 mono- or di-methylation of 23S rRNA adenosine (A) 2085 (corresponding to E. coli A2058, leading to resistance towards MLS antibiotics)^{6,7}, 23S rRNA from S. aureus grown in erythromycin was hardly stimulatory. In contrast, 23S rRNA from resistant S. aureus not grown in erythromycin and also from E. coli (including EHEC) but not 16S rRNA of both activated TLR23479^{-/-} macrophages to normal degrees (FIG. 2g; FIG. 2.1e, f). Moreover, overexpression of ermB and ermC (the latter being subcloned from cDNA of an erythromycin grown S. aureus isolate) conferred in E. coli and B. subtilis strains erythromycin resistance and ablated 23S rRNA stimulatory activity towards TLR23479^{-/-} macrophages (FIG. 2h). These data indicated that resistance to MLS group antibiotics (including erythromycin) mediated by site-specific methylation (targeting A2085 in S. aureus and A2058 in E. coli 23S rRNA) rendered 23S rRNA non-stimulatory.

### Example 3: Identification of the Minimal Stimulatory Sequence

To address the immune stimulatory activity of 23S rRNA in more detail, we designed three ORNs as analogues of S. aureus 23S rRNA segments each of which carries an A in its centre that becomes methylated constitutively or under growth restriction to modulate the docking of protein synthesis cofactors or antibiotics. The three ORNs named SaI, SaII, and SaIII represented S. aureus A1662 (E. coli A1616, methylation of which promotes fitness²⁰), S. aureus A2530 (E. coli A2503, targeted by chloramphenicol, florfenicol, and clindamycin resistance RNA methyltransferase²¹), as well as S. aureus A2085^{6,7} (E. coli A2058), respectively (Table 1).

Only SaIII (which mirrors S. aureus A2085) activated TLR23479^{-/-} cells (FIG. 3a). PDCs recognised SaIII via TLR7, but this activity was lost with 3′-terminal deletion (FIG. 3.1a). ORNs resulting from deletions of 3′- and 5′-termini (SaIIId3, SaIIId5, Sa23) equally activated TLR23479^{-/-} cDCs (FIG. 3b), whereas preincubation of S. aureus RNA or of ORN Sa23 with an antisense SaIII RNA strand (SaIIIas) abrogated the stimulatory activity (FIG. 3c). These results indicated single strand structure as well as singularity of the stimulatory activity within the bacterial transcriptome.

Successive terminal deletions towards a 12-mer ORN (Sa12, Table 1) led to sequences that are identical in S. aureus and E. coli 23S rRNAs. Length dependent reduction of stimulatory capacity could largely be compensated by terminal fill-ups (Sa12A19, FIG. 3d)²². Sa12 N6-methylated at A6 (corresponding to S. aureus A2085 and mimicking erm-methylated 23S rRNA) lacked stimulatory capacity, whereas A7 N6-methylation merely caused partial reduction (FIG. 3e). Mutations at the termini of Sa12 revealed “ACGGAAAGACC” (SEQ ID NO: 35) as the minimal stimulatory segment (Sa12t5b) because further mutation of the 5′ end (Sa12t5c) or the 3′ end (Sa12t3b) abrogated the stimulatory activity (FIG. 3f; FIG. 3.1.b; Table 1).

Coincidentally, the macrolide, lincosamide, and streptogramin (MLS)-group antibiotics (such as erythromycin) binding site is contained in this motif that carries A2085 in S. aureus (or A2058 in E. coli) 23S rRNA, the N6 methylation or mutation of which confers resistance to MLS antibiotics^{6,7}. Hence, 23S rRNA from clinical isolates of erythromycin-resistant S. aureus and from erythromcin resistance methyltransferase (erm) B/C overexpressing bacteria, as well as synthetic oligoribonucleotides (ORNs) carrying N6 methylated A or a guanosine (G) as A2085/2058 replacements failed to stimulate TLR13. Our results thus demonstrate that sequence-specific 23S rRNA modifications render bacteria resistant to certain naturally occurring antibiotics and to immune recognition by TLR13.

On the other hand, Sa12 derivatives mimicking eukaryotic 28S rRNA or specific 23S rRNA mutations that render bacteria resistant to MLS antibiotics (S. aureus 23S rRNA A2085G, mimicked by A6G/Sa12s6) failed to stimulate bone marrow cells (FIG. 3g, Table 1)^{7,19,23}. These findings suggested that molecular mechanisms rendering bacteria resistant to naturally occurring antibiotics also impede MyD88 dependent host recognition by an ill-defined endosomal TLR.

In addition, our data unravel an unanticipated link between antibiotic resistance and evasion from TLR13 recognition, since 23S rRNA modifications generating resistance towards MLS antibiotics also camouflaged bacteria from TLR13 recognition. MLS antibiotics producing bacteria such as Saccharopolyspora erythraea were possibly first to express erms (to resist their own antibiotics)⁶. Erm expression plasmids might have been acquired from S. erythraea by staphylococci, pneumococci, and mycobacteria^{6,24}. As resistance trait spinoff, the pathogenic recipients gained invisibility to TLR13. We therefore speculate that widespread ancient antibiotic resistance²⁵ has subverted TLR13 driven antibacterial immune resistance. This may explain why TLR13 expression has been abandoned in certain mammalian species, including human.

### Example 4

A TLR8^{-/-} cell analysis ruled out the involvement of TLR8 (not shown). We thus focussed at TLR13-specific siRNA-driven suppression of TLR13 mRNA accumulation that impaired the recognition of hiSa or stimulatory ORNs such as SaIII (FIG. 1g; FIG. 4a; FIG. 4.1a). Furthermore, ectopic expression of TLR13 but not of CD14, TLR3, -7, -8, -9 or -12 conferred to HEK293 cell responsiveness towards challenge with hiSa or the ORNs SaIII, Sa23, Sa17, or Sa12 (FIG. 4b-d; FIG. 4.1b, c). Other nucleotides such as RNA40 (TLR7 ligand) or CpG-DNA (TLR9 ligand) were inactive (FIG. 4e). In vivo application of a phosphorothioate Sa19 variant (Sa19PSO) triggered systemic pro-inflammatory cytokine release similar to that elicited by the PSO-CpG-oligodeoxynucleotide 1668 (FIG. 4f; FIG. 4.1d).

### REFERENCES

a. Kawai, T. & Akira, S. Toll-like receptors and their crosstalk with other innate receptors in infection and immunity. Immunity 34, 637-650 (2011).
b. Brightbill, H. D. et al. Host defense mechanisms triggered by microbial lipoproteins through toll-like receptors. Science 285, 732-736. (1999).
c. Hemmi, H. et al. A Toll-like receptor recognizes bacterial DNA. Nature 408, 740-745. (2000).
d. Karikó, K., Buckstein, M., Ni, H. & Weissman, D. Suppression of RNA recognition by Toll-like receptors: the impact of nucleoside modification and the evolutionary origin of RNA. Immunity 23, 165-175 (2005).
e. Mancuso, G. et al. Bacterial recognition by TLR7 in the lysosomes of conventional dendritic cells. Nat Immunol 10, 587-594 (2009).
f. Skinner, R. H. & Cundliffe, E. Dimethylation of adenine and the resistance of Streptomyces erythraeus to erythromycin. J Gen Microbiol 128, 2411-2416 (1982).
g. Weisblum, B. Erythromycin resistance by ribosome modification. Antimicrob Agents Chemother 39, 577-585 (1995).
h. Girardin, S. E. et al. Nod2 is a general sensor of peptidoglycan through muramyl dipeptide (MDP) detection. J Biol Chem 278, 8869-8872 (2003).
i. Spiller, S. et al. TLR4-induced IFN-gamma production increases TLR2 sensitivity and drives Gram-negative sepsis in mice. J Exp Med 205, 1747-1754 (2008).
j. Ruland, J. CARD9 signaling in the innate immune response. Ann N Y Acad Sci 1143, 35-44 (2008).
k. Muñoz-Planillo, R., Franchi, L., Miller, L. S. & Núñez, G. A critical role for hemolysins and bacterial lipoproteins in Staphylococcus aureus-induced activation of the Nlrp3 inflammasome. J Immunol 183, 3942-3948 (2009).
l. Tschopp, J. & Schroder, K. NLRP3 inflammasome activation: The convergence of multiple signalling pathways on ROS production? Nat Rev Immunol 10, 210-215 (2010).
m. Blasius, A. L. & Beutler, B. Intracellular toll-like receptors. Immunity 32, 305-315 (2010).
n. Brinkmann, M. M. et al. The interaction between the ER membrane protein UNC93B and TLR3, 7, and 9 is crucial for TLR signaling. J Cell Biol 177, 265-275 (2007).
o. Conrad, M. L. et al. Maternal TLR signaling is required for prenatal asthma protection by the nonpathogenic microbe Acinetobacter lwoffii F78. J Exp Med 206, 2869-2877 (2009).
p. Luber, C. A. et al. Quantitative proteomics reveals subset-specific viral recognition in dendritic cells. Immunity 32, 279-289 (2010).
q. Heil, F. et al. Species-specific recognition of single stranded RNA via Toll-like receptor 7 and 8. Science 303, 1526-1529 (2004).
r. Deshmukh, S. D. et al. Macrophages recognize streptococci through bacterial single-stranded RNA. EMBO Rep 12, 71-76 (2011).
s. Vester, B. & Douthwaite, S. Macrolide resistance conferred by base substitutions in 23S rRNA. Antimicrob Agents Chemother 45, 1-12 (2001).
t. Sergiev, P. V., Serebryakova, M. V., Bogdanov, A. A. & Dontsova, 0. A. The ybiN gene of Escherichia coli encodes adenine-N6 methyltransferase specific for modification of A1618 of 23 S ribosomal RNA, a methylated residue located close to the ribosomal exit tunnel. J Mol Biol 375, 291-300 (2008).
u. Long, K. S., Poehlsgaard, J., Kehrenberg, C., Schwarz, S. & Vester, B. The Cfr rRNA methyltransferase confers resistance to Phenicols, Lincosamides, Oxazolidinones, Pleuromutilins, and Streptogramin A antibiotics. Antimicrob Agents Chemother 50, 2500-2505 (2006).
v. Hornung, V. et al. Sequence-specific potent induction of IFN-alpha by short interfering RNA in plasmacytoid dendritic cells through TLR7. Nat Med 11, 263-270 (2005).
w. Klinge, S., Voigts-Hoffmann, F., Leibundgut, M., Arpagaus, S. & Ban, N. Crystal structure of the eukaryotic 60S ribosomal subunit in complex with initiation factor 6. Science 334, 941-948 (2011).
x. Buriánková, K. et al. Molecular basis of intrinsic macrolide resistance in the Mycobacterium tuberculosis complex. Antimicrob Agents Chemother 48, 143-150 (2004).
y. D'Costa, V. M. et al. Antibiotic resistance is ancient. Nature 477, 457-461 (2011).
z. Spiller, S. et al. Cellular recognition of trimyristoylated peptide or enterobacterial lipopolysaccharide via both TLR2 and TLR4. J Biol Chem 282, 13190-13198 (2007).
aa. Lauterbach, H. et al. Mouse CD8alpha+ DCs and human BDCA3+ DCs are major producers of IFN-lambda in response to poly IC. J Exp Med 207, 2703-2717 (2010).
bb. Fuchs, S., Pané-Farré, J., Kohler, C., Hecker, M. & Engelmann, S. Anaerobic gene expression in Staphylococcus aureus. J Bacteriol 189, 4275-4289 (2007).
cc. Trieu-Cuot, P., Carlier, C., Poyart-Salmeron, C. & Courvalin, P. Shuttle vectors containing a multiple cloning site and a lacZ alpha gene for conjugal transfer of DNA from Escherichia coli to gram-positive bacteria. Gene 102, 99-104 (1991).
dd. Wiese, M. et al. Small interfering RNA (siRNA) delivery into murine bone marrow-derived macrophages by electroporation. J Immunol Methods 353, 102-110 (2010).
ee. Fancke B, Suter M, Hochrein H, O'Keeffe M. M-CSF: a novel plasmacytoid and conventional dendritic cell poietin. Blood. 2008 Jan. 1; 111(1):150-9.
ff. Vremec D, O'Keeffe M, Hochrein H, Fuchsberger M, Caminschi I, Lahoud M, Shortman K. Production of interferons by dendritic cells, plasmacytoid cells, natural killer cells, and interferon-producing killer dendritic cells. Blood. 2007 Feb. 1; 109(3):1165-73.

1. A pharmaceutical composition for modulating Toll-like receptor-13 (TLR-13) or Toll-like receptor-13 (TLR-13)-expressing cells in a subject, the pharmaceutical composition comprising a nucleic acid sequence selected from the group consisting of: SEQ ID NO:1, SEQ ID NO: 13, SEQ ID NO:15, and a or a functional variant thereof, wherein the functional variant thereof is capable of: (1) activating TLR-13; (2) activating TLR-13 expressing cells; or (3) is capable of stimulating an immune response in a non-primate subject.

2. The pharmaceutical composition of claim 1, wherein the functional variant of SEQ ID NO: 1 is selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO:12.

3. The pharmaceutical composition of claim 1, wherein the functional variant of SEQ ID NO: 13 is SEQ ID NO:14.

4. The pharmaceutical composition of claim 1, wherein the nucleic acid sequence is selected from SEQ ID NO: 2 and SEQ ID NO: 10.

5. The pharmaceutical composition of claim 1, wherein the nucleic acid sequence is from SEQ ID NO: 2.

6. The pharmaceutical composition of claim 1, wherein the nucleic acid sequence is SEQ ID NO: 10.

7. The pharmaceutical composition of claim 1 further comprising an immunostimulant or an immunosuppressant.

8. The pharmaceutical composition of claim 1, wherein the composition is an adjuvant.

9. A method for treating or vaccinating against a bacterial infection in a subject, comprising administering to the subject a pharmaceutical agent comprising SEQ ID NO:1, SEQ ID NO: 13, SEQ ID NO:15, or a functional variant thereof, wherein the functional variant thereof is capable of: (1) activating TLR-13; (2) activating TLR-13 expressing cells; or (3) is capable of stimulating an immune response in a non-primate subject.

10. The method of claim 9, wherein the bacterial infection is caused by a Gram-positive or a Gram-negative bacterium.

11. The method of claim 10, wherein the bacterial infection induced septic syndrome.

12. The method of claim 9, wherein the functional variant of SEQ ID NO:1 is selected from SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO:10, SEQ ID NO: 11, and SEQ ID NO:12.

13. The method of claim 9, wherein the functional variant of SEQ ID NO:13 is SEQ ID NO:14.

14. The method of claim 10, wherein bacterial infection is caused by a Gram-positive or a Gram-negative bacterium resistant to erythromycin.

15. The method of claim 10, wherein bacterial infection is caused by a Gram-positive bacterium resistant to erythromycin.

16. The method of claim 10, wherein the bacterial infection is caused by a Gram-positive or a Gram-negative bacterium resistant to one or more antibiotics of the macrolide, lincosamide, and streptogramin (MLS) group.

17. The method of claim 10, wherein the Gram positive bacterium resistant to erythromycin is Staphylococcus aureus or Streptococcus pneumoniae.
